(19) United States
(12) Patent Application Publication
(10) Pub. No.: US 2025/0356487 A1
(43) Pub. Date: Nov. 20, 2025

(54) SYSTEMS AND METHODS FOR COLLABORATIVE REVIEW OF ELECTRONIC IMAGES IN DIGITAL PATHOLOGY

(71) Applicant: Paige.AI, Inc., New York, NY (US)

(72) Inventors: James KIM, Bayside, NY (US); Csaba BARTFAI, Szeged (HU); Kornel PALKOVICS, Budapest (HU); Peter ARADI, Budapest (HU); Joe MARBACH, Raleigh, NC (US); Danielle GORTON, Beacon, NY (US)

(21) Appl. No.: 19/210,428

(22) Filed: May 16, 2025

Related U.S. Application Data
(60) Provisional application No. 63/648,894, filed on May 17, 2024.

(51) Int. Cl. G06T 7/00; G06F 3/14; G06T 19/20; G16H 70/60; H04L 65/1089; H04L 65/401

(52) U.S. Cl. CPC G06T 7/0012; G06F 3/1454; G06T 19/20; G16H 70/60; H04L 65/1089; H04L 65/4015; G06T 2200/24; G06T 2207/10056; G06T 2207/30024; G06T 2207/30068; G06T 2210/41; G06T 2219/004; G06T 2219/2016

(57) ABSTRACT

The present disclosure provides a method for collaborative review of digital pathology images. The method may include initiating a collaboration session including a plurality of practitioners on an electronic network, receiving an indication of one or more digital pathology images to be reviewed in the collaboration session from a first session instance associated with a first practitioner, providing the indication to at least a second session instance associated with a second practitioner, receiving viewing data including zoom level, viewport rotation, and cursor coordinates associated with the first session instance, the viewing data not including video data, and providing the viewing data to at least the second session instance.

### CROSS-REFERENCE TO RELATED APPLICATIONS

This application claims priority to U.S. Provisional Application No. 63/648,894, filed May 17, 2024, the entire disclosure of which is incorporated herein by reference in its entirety.

### FIELD OF DISCLOSURE

Various embodiments of the present disclosure pertain generally to digital pathology and collaborative review of medical images. More specifically, particular embodiments of the present disclosure relate to systems and methods for enabling real-time collaborative review of high-resolution digital pathology images among multiple practitioners.

### BACKGROUND

Digital pathology has revolutionized the field of pathology by enabling the digitization and remote analysis of tissue samples. This technology has greatly enhanced collaboration among pathologists and improved diagnostic accuracy. However, current methods for collaborative review of digital pathology images face several challenges that limit their effectiveness.

Existing approaches to collaborative review often rely on screen sharing applications, which have inherent limitations. These applications typically transmit low-resolution images, resulting in a loss of critical detail that is essential for accurate diagnosis. Additionally, screen sharing can introduce significant lag time and consume substantial bandwidth, making real-time collaboration difficult and potentially frustrating for users.

Another issue with current collaborative review systems is the difficulty in precisely indicating specific areas of interest on a digital slide. Pathologists often need to draw attention to particular regions or cellular structures, but existing tools may not provide an intuitive or accurate way to do so. This can lead to miscommunication and potential errors in diagnosis or interpretation.

Furthermore, many collaborative review platforms do not adequately support the complex workflow of pathology case review. Pathologists frequently need to examine multiple slides per case, compare different stains, and refer to patient history or previous diagnoses. Current systems may not seamlessly integrate these various aspects of the review process, forcing pathologists to switch between multiple applications or rely on cumbersome workarounds.

The increasing use of artificial intelligence (AI) in pathology also presents new challenges for collaborative review. While AI algorithms can assist in detecting and classifying certain features, there is a need for systems that can effectively integrate AI findings into the collaborative review process, allowing pathologists to discuss and validate these results in real-time.

Lastly, existing collaborative review systems often lack features for efficient case management and organization. As the volume of digital pathology cases grows, pathologists require tools to prioritize, tag, and sort cases for review, especially in scenarios involving multiple reviewers or institutions.

These limitations in current collaborative review systems for digital pathology highlight the need for improved solutions that can address these challenges and enhance the efficiency and accuracy of pathological diagnoses.

The foregoing general description and the following detailed description are exemplary and explanatory only and are not restrictive of the disclosure. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY

According to certain aspects of the present disclosure, computer implemented systems and methods are disclosed for collaborative review of electronic images in digital pathology, including: initiating a collaboration session including a plurality of practitioners on an electronic network; receiving an indication of one or more digital pathology images to be reviewed in the collaboration session from a first session instance associated with a first practitioner of the plurality of practitioners; providing the indication of the one or more digital pathology images to at least a second session instance associated with a second practitioner of the plurality of practitioners; viewing data may include zoom level, viewport rotation, and cursor coordinates associated with the first session instance, the viewing data not including video data; and providing the viewing data to at least the second session instance.

In some aspects, the techniques described herein relate to a method, wherein the viewing data further comprises annotation data, and the second session instance displays the cursor coordinates and annotation data, but not the zoom level or viewport rotation. In some aspects, the techniques described herein relate to a method, wherein the annotation data may include real-time updates of annotations made by the first practitioner during the collaboration session. The method may enable real-time collaboration between practitioners viewing the same high-resolution digital pathology images without sharing the entire image data, facilitating efficient remote consultation and diagnosis.

In some aspects, the techniques described herein relate to a method, wherein the viewing data including zoom level, viewport rotation, and cursor coordinates may be used to reconstruct a view of the first session instance within the second session instance.

In some aspects, the techniques described herein relate to a method, wherein each practitioner of the plurality of practitioners is associated with an avatar displayed within the collaboration session, and further including: receiving a selection of a second avatar in the first session instance, the second avatar being associated with the second session instance; and in response to the selection, providing data indicative of zoom level, viewport rotation, and cursor coordinates associated with the second session instance to the first session instance.

In some aspects, the techniques described herein relate to a method, further including: displaying a case list comprising multiple cases available for review in the collaboration session; and receiving a selection of a case from the case list, from the first session instance, to be reviewed in the collaboration session.

In some aspects, the techniques described herein relate to a method, wherein the case list may be filtered based on tags associated with each case, the tags including at least one of date labels, tissue type labels, or diagnostic labels

According to another aspect of the present disclosure, a system for collaborative review of digital pathology images is disclosed, the system including: a processor and a memory storing instructions that, when executed by the processor, cause the system to execute the instructions to perform operations. The operations may include: initiating a collaboration session including a plurality of practitioners; receiving, from a first client device, an indication of one or more digital pathology images to be reviewed; transmitting the indication to at least a second client device; receiving, from the first client device, viewing data comprising zoom level, viewport rotation, and cursor coordinates, the viewing data not comprising video data; and transmitting the viewing data to at least the second client device.

In some aspects, the techniques described herein relate to a method, wherein the viewing data may further include annotation data, and the operations may further include transmitting the annotation data to at least the second client device for display of cursor coordinates and annotation data, but not the zoom level or viewport rotation. The annotation data may include real-time updates of annotations made by a first practitioner during the collaboration session.

In some aspects, the techniques described herein relate to a system, wherein the viewing data transmitted to at least the second client device may be used to reconstruct a view of the first client device within the second client device.

In some aspects, the techniques described herein relate to a system, wherein the operations may further include associating each practitioner of the plurality of practitioners with an avatar displayed within the collaboration session, receiving, from the first client device, a selection of a second avatar associated with the second client device, and in response to the selection, transmitting data indicative of zoom level, viewport rotation, and cursor coordinates associated with the second client device to the first client device.

The operations may further include transmitting a case list comprising multiple cases available for review in the collaboration session to the first client device and the second client device, and receiving a selection of a case from the case list to be reviewed in the collaboration session. The case list may be filtered based on tags associated with each case, the tags comprising at least one of date labels, tissue type labels, or diagnostic labels.

According to certain aspects of the present disclosure, a non-transitory computer-readable storage medium storing instructions that when executed by a processor, the instructions cause the processor to perform a method for collaborative review of digital pathology images, the method including: initiating a collaboration session including a plurality of practitioners on an electronic network; receiving an indication of one or more digital pathology images to be reviewed in the collaboration session from a first session instance associated with a first practitioner of the plurality of practitioners; providing the indication of the one or more digital pathology images to at least a second session instance associated with a second practitioner of the plurality of practitioners; receiving viewing data including zoom level, viewport rotation, and cursor coordinates associated with the first session instance, the viewing data not comprising video data; and providing the viewing data to at least the second session instance.

In some aspects, the techniques described herein relate to a non-transitory computer-readable medium, wherein the viewing data further comprises annotation data, and the method further including: transmitting the annotation data to the second session instance for display of cursor coordinates and annotation data, but not the zoom level or viewport rotation.

In some aspects, the techniques described herein relate to a non-transitory computer-readable medium, wherein the annotation data comprises real-time updates of annotations made by the first practitioner during the collaboration session.

In some aspects, the techniques described herein relate to a non-transitory computer-readable medium, wherein the method may further include: associating each practitioner of the plurality of practitioners with an avatar displayed within the collaboration session; receiving, from the second session instance, a selection of an avatar representing the first practitioner; and in response to the selection, transmitting data indicative of zoom level, viewport rotation, and cursor coordinates associated with a client device of the first practitioner to the client device associated with the second practitioner.

In some aspects, the techniques described herein relate to a non-transitory computer-readable medium, wherein the method may further include: providing a case list comprising multiple cases available for review in the collaboration session; and receiving a selection of a case from the case list to be reviewed in the collaboration session.

In some aspects, the techniques described herein relate to a non-transitory computer-readable medium, wherein the case list may be filtered based on tags associated with each case, the tags including at least one of date labels, tissue type labels, or diagnostic labels.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 depicts a user interface of a digital pathology collaboration system, according to techniques discussed herein.

FIG. 2 depicts a collaborative digital pathology viewing interface with interactive elements, as described herein.

FIG. 3 depicts a collaborative digital pathology review interface with search and case management features, in accordance with techniques discussed herein.

FIG. 4 depicts a digital pathology viewing interface displaying a microscope slide image and case list, as described in embodiments herein.

FIG. 5 depicts a flowchart of a method for collaborative review in digital pathology, according to techniques described herein.

FIG. 6 depicts a block diagram of an exemplary environment for processing and sharing digital pathology images, according to techniques discussed herein.

FIG. 7 depicts a flow diagram of a training system for machine learning models in digital pathology, according to techniques discussed herein.

FIG. 8 depicts a block diagram of a computing device that may be used in digital pathology collaboration systems discussed herein.

Notably, for simplicity and clarity of illustration, certain aspects of the figures depict the general configuration of the various embodiments. Descriptions and details of well-known features and techniques may be omitted to avoid unnecessarily obscuring other features. Elements in the figures are not necessarily drawn to scale; the dimensions of some features may be exaggerated relative to other elements to improve understanding of the example embodiments.

### DETAILED DESCRIPTION

Reference will now be made in detail to the exemplary embodiments of the present disclosure, examples of which are illustrated in the accompanying drawings. Wherever possible, the same reference numbers will be used throughout the drawings to refer to the same or like parts.

The systems, devices, and methods disclosed herein are described in detail by way of examples and with reference to the figures. The examples discussed herein are examples only and are provided to assist in the explanation of the apparatuses, devices, systems, and methods described herein. None of the features or components shown in the drawings or discussed below should be taken as mandatory for any specific implementation of any of these devices, systems, or methods unless specifically designated as mandatory.

Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

Techniques described in the current disclosure may utilize systems and methods described in U.S. application Ser. No. 18/061,837, U.S. application Ser. No. 18/295,577, U.S. application Ser. No. 18/630,072, and U.S. application Ser. No. 18/920,046, all of which are incorporated herein by reference.

As used herein, the term “exemplary” is used in the sense of “example,” rather than “ideal.” Moreover, the terms “a” and “an” herein do not denote a limitation of quantity, but rather denote the presence of one or more of the referenced items.

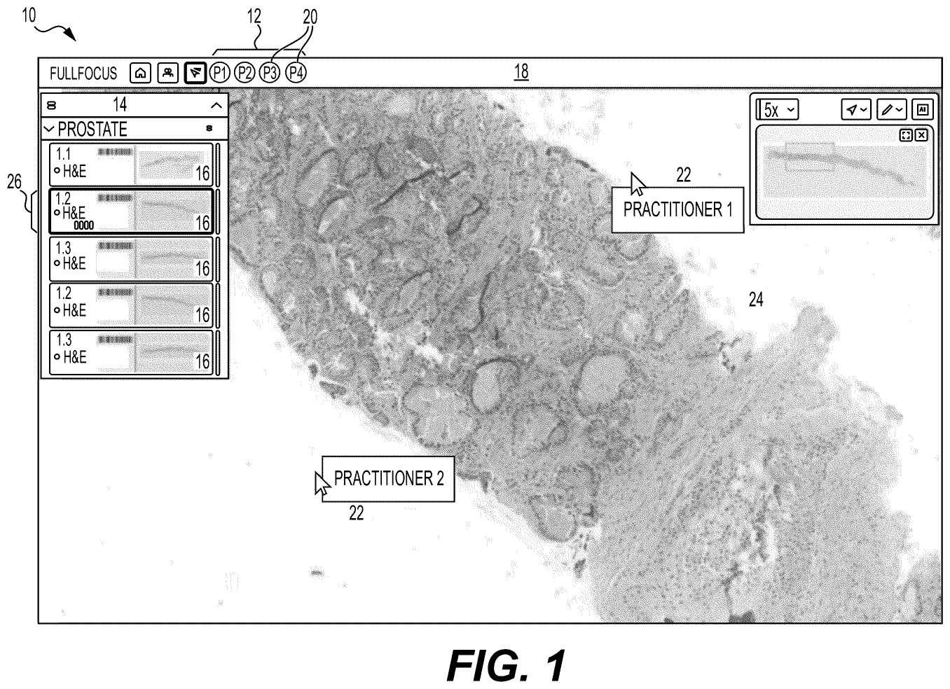

The digital pathology collaboration system, as illustrated in FIG. 1, provides a user interface 10 that enables real-time collaboration among pathologists. FIG. 1 depicts several key components of the system that facilitate collaborative review of digital pathology slides.

Patient information 18 may be displayed on the interface. This information may be presented flexibly in various locations on the interface, such as along the top edge, on either side, or at the bottom of the display. The specific positioning of patient information 18 can be adapted based on practitioner preferences or the particular layout requirements of the collaborative review session, allowing practitioners to maintain access to relevant clinical data while focusing on the digital pathology images.

The patient information 18 displayed on the user interface 10 may include a wide range of data relevant to the case under review. In some aspects, this information may comprise, among other things, name, age/date of birth, sex, and/or medical record number. The specific patient information 18 displayed may be customized based on the type of case, medical specialty, and individual practitioner preferences. The system may allow practitioners to configure which categories of information are shown and in what order, enabling efficient access to the most relevant data for each collaborative review session. The system may allow practitioners to select their preferred positioning of the elements on the user interface, enabling customization of the interface layout to suit individual workflows and preferences. This flexible positioning of patient information 18, and other elements, may help optimize the use of screen real estate while ensuring that important clinical context remains readily available during collaborative review sessions.

A practitioner list 12 may be displayed on the user interface 10. The practitioner list 12 may show practitioner avatar(s) 20 representing the practitioners participating in the collaboration session. This feature allows users to quickly identify and interact with their colleagues during the review process.

The practitioner avatar(s) 20 may take on various forms and representations to suit individual preferences and system requirements. In some aspects, the practitioner avatar(s) 20 may be customized to appear as any shape, image, or graphical element. For example, a practitioner's avatar 20 may be a photograph, an illustrated character, a logo, or any other visual representation chosen by the user or assigned by the system. Additionally, in some implementations, the system may support using just the practitioner's initials as their practitioner avatar 20, providing a simple text-based option for identification. The avatar 20 might also display live video of the practitioner automatically cropped to the practitioner's head. This flexibility in practitioner avatar 20 representation may allow for easy differentiation between practitioners and accommodate various organizational or personal preferences for visual identification within the collaborative environment.

In some aspects, the practitioner list 12 may be displayed in various locations on the user interface 10. For example, practitioner list 12 may be positioned at the top of the user interface 10, providing a prominent location for users to quickly identify and interact with their colleagues. Alternatively, practitioner list 12 may be displayed at the bottom of the user interface 10, allowing for easy access while maximizing the viewing area for digital pathology images. In other implementations, practitioner list 12 may be positioned along either the left or right side of the interface, providing a vertical arrangement of practitioner avatars 20. The system may allow practitioners to select their preferred positioning of the practitioner list 12, enabling customization of the interface layout to suit individual workflows and preferences. This flexible positioning of practitioner list 12 may help optimize the use of screen real estate while ensuring that information about participating colleagues remains readily available during collaborative review sessions.

A slide tray 14 may be visible on the interface. The slide tray 14 may contain thumbnail images 16 of multiple pathology slides available for review within the current case.

The main viewing area 24 of the interface may display a high-resolution digital pathology slide. This central component allows practitioners to examine tissue samples in detail while maintaining access to the collaborative features provided by the practitioner list 12, slide tray 14, and avatar indicators 22.

The main viewing area 24 may incorporate avatar indicators 22 that represent the cursor locations of other practitioners participating in the collaborative session. These avatar indicators 22 may be displayed as small graphical elements overlaid on the digital pathology slide image. In some aspects, the avatar indicators 22 may take on various forms to uniquely identify each practitioner, such as displaying the practitioner's initials or the practitioner's full name. The avatar indicators 22 may also display a photo or automatically cropped video of the practitioner. For example, the video of the practitioner may be cropped to the practitioner's face. As the practitioner speaks, text may be automatically transcribed and shown in proximity to the associated avatar indicator 22. One function of these avatar indicators 22 is to visually indicate the current cursor position of each practitioner within the digital pathology slide, allowing other participants to easily track where each practitioner is focusing their attention in real-time. These indicators enable efficient collaboration by showing precisely where each practitioner is examining the tissue sample during the review session.

As used herein, the terms “cursor coordinates,” “cursor location,” and “cursor position” may be used interchangeably to refer to the spatial position of a cursor within the digital pathology image viewing area. These terms may indicate the x and y coordinates, or other suitable positional data, that specify where a practitioner's cursor is located on the digital pathology slide at any given time during the collaborative review session.

The positioning of the avatar indicators 22 within the main viewing area 24 may dynamically update in real-time as practitioners move their cursors, tap with a finger, or gaze if eye tracking, across the digital pathology slide. This real-time updating may enable participants to visually track the areas of interest being examined by their colleagues during the collaborative review session. In some implementations, the system may use interpolation techniques to smooth the movement of avatar indicators 22, providing a more fluid visual representation of cursor movements.

The main viewing area 24 may also include functionality that allows practitioners to interact with the avatar indicators 22. For example, hovering over or selecting an avatar indicator(s) 22 may display additional information about the corresponding practitioner, such as their name or role. In some embodiments, hovering or clicking on avatar indicator(s) 22 may also provide and/or synchronize the zoom level, cursor coordinates, and/or viewport rotation of the practitioner.

Zoom level may refer to the magnification level at which the digital pathology image is being viewed. The zoom level may be represented as a numerical value, percentage, or scale factor that indicates how much the image has been magnified relative to its original size. In digital pathology, practitioners frequently adjust zoom levels to examine cellular details at different magnifications, similar to changing objective lenses on a traditional microscope. The system may support multiple predefined zoom levels (such as 1×, 2×, 5×, 10×, 20×, 40×) or allow continuous zooming for precise examination of tissue structures. When synchronized between practitioners during collaborative sessions, the zoom level ensures all participants can view the same level of detail simultaneously.

As used herein, the term “viewport rotation” may refer to the angular orientation or rotational position of the visible area of a digital pathology image displayed on a user's screen. Viewport rotation may allow a practitioner to adjust the angle at which the tissue sample is viewed, potentially revealing different perspectives or alignments of cellular structures. The rotation may be measured in degrees and may be adjustable through user input such as mouse movements, touch gestures, or keyboard commands. In some implementations, viewport rotation may be synchronized across multiple users' displays to ensure all practitioners are viewing the sample from the same orientation during collaborative sessions.

In some aspects, clicking on or otherwise selecting avatar indicator(s) 22 may initiate a “follow” mode, where the viewing perspective of the clicking practitioner automatically synchronizes with that of the selected colleague, which may synchronize zoom level, viewport rotation, and/or cursor coordinates. Such a mode might not require actual video data be sent to other practitioner views, which may improve synchronization lag, reduce data usage, and allow for various degrees of partial synchronization, which is discussed further herein.

By transmitting only the essential viewing parameters rather than full video streams, the system can achieve more efficient real-time collaboration even in bandwidth-constrained environments. This approach significantly reduces network load compared to traditional screen sharing methods, as only small data packets containing coordinates and viewing parameters need to be transmitted rather than continuous video frames. The reduced data requirements may also enable more responsive synchronization between practitioners, minimizing latency that could otherwise impede collaborative analysis. Additionally, this parameter-based synchronization approach provides greater flexibility in how practitioners can customize their collaborative experience, allowing them to selectively synchronize specific aspects of the viewing experience while maintaining independent control over other parameters. For example, a practitioner might choose to synchronize with a colleague's cursor position and annotations while maintaining their own preferred zoom level or viewport rotation, creating a more personalized yet still collaborative viewing experience.

In some cases, the digital pathology collaboration system may include additional controls for magnification, navigation, and other viewing functions. These controls may be integrated into the user interface 10 to enhance the practitioners' ability to analyze the digital pathology slides effectively while collaborating with their colleagues in real-time.

In some aspects, when a practitioner joins a live collaboration session, they may be presented with additional interactive elements and information to facilitate seamless collaboration. The system may display the practitioner list 12, allowing the joining practitioner to see a list of other practitioners who have already joined the session. This feature may provide immediate context about the participants involved in the collaborative review.

The digital pathology collaboration system may offer customizable visibility options for cursor tracking. In some implementations, practitioners may have the ability to toggle, show, and/or hide other cursors visible in the session. This flexibility may allow practitioners to adjust the level of visual information displayed in the main viewing area 24 according to their preferences or the specific requirements of the collaborative task at hand.

The slide tray 14 may display thumbnail images 16 of all slides available for a given case. This comprehensive view may allow practitioners to quickly assess the scope of the case and navigate between different slides efficiently. In some implementations, the slide tray 14 may organize the thumbnail images 16 in a scrollable or paginated format to accommodate cases with a large number of slides. The system may also provide options for practitioners to customize the arrangement or grouping of thumbnails within the slide tray 14 based on their preferences or workflow requirements. In a collaborative environment, in “follow” mode, the slide tray may be altered to reflect the view of the practitioner being followed.

The slide currently under examination in the collaborative session may be visually distinguished within the slide tray 14. For example, the thumbnail image 16 corresponding to the active slide 26 may be highlighted with a colored border, increased brightness, or a subtle glow effect. Alternatively, the system may apply a marker or icon to the active slide's thumbnail, such as a small flag or star. This visual differentiation may help practitioners quickly identify which slide is currently being viewed in the main viewing area 24, especially when rapidly switching between multiple slides during the collaborative review process.

In some aspects, the slide tray 14 may include additional interactive features to enhance navigation and organization. For instance, practitioners may have the ability to drag and reorder thumbnails within the slide tray 14, allowing them to arrange slides in a preferred sequence for review. The slide tray 14 may also automatically reorganize based upon whether the slides have previously been viewed, based upon presence of cancer or other possible pathology, as determined by a machine learning algorithm.

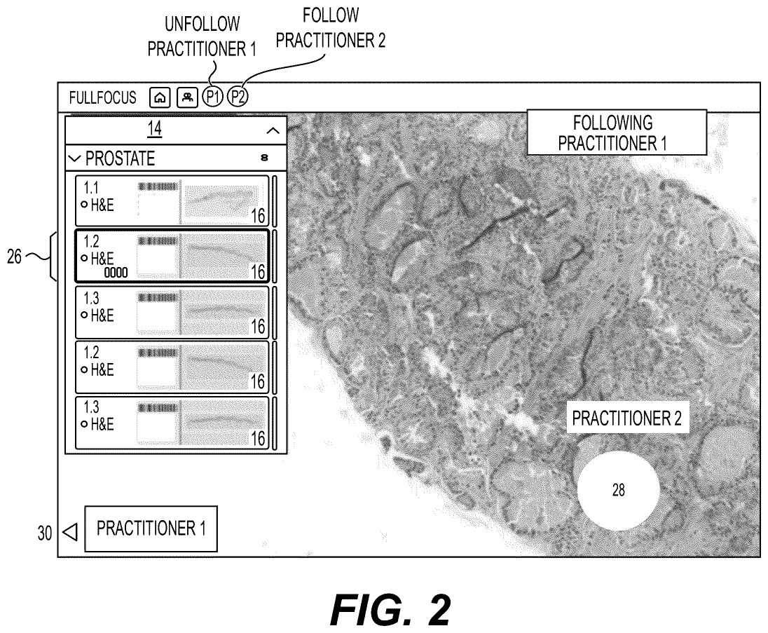

The digital pathology collaboration system may include interactive elements that facilitate real-time collaboration between practitioners, as illustrated in FIG. 2. These elements may enhance the ability of practitioners to communicate and synchronize their viewing activities during collaborative sessions. In some cases, as can be seen in FIG. 2, the system may display an active cursor indicator 28 within the visible area of the digital pathology image. The active cursor 28 may represent the current position of a collaborating practitioner's cursor on the shared digital slide. This feature may allow practitioners to visually track the focus area of their colleagues in real-time. The system may also include an offscreen cursor indicator 30. In some cases, when a practitioner's cursor moves outside the current field of view, the offscreen cursor indicator may appear at the edge of the visible area. The offscreen cursor indicator 30 may provide a visual cue to other practitioners about the direction and approximate location of their colleague's cursor when it is not directly visible on the screen. The offscreen cursor indicator 30 may provide additional context when practitioners are viewing different areas of the slide. For example, if one practitioner's cursor moves beyond the visible area of another practitioner's viewport, the offscreen cursor indicator 30 may appear, helping to maintain awareness of collaborative activities even when direct visual contact with the cursor is lost.

The system may in addition include a follow control that enables practitioners to synchronize their view with another participant's view of the slide. When activated, the follow control may allow automatic viewport synchronization, matching the zoom level, rotation, and/or position of the followed practitioner's view. This feature may facilitate guided tours or collaborative analysis of specific regions of interest on the digital pathology slide. In some cases, a practitioner may click on the practitioner avatar 20 or offscreen cursor indicator 30 of another practitioner to initiate the follow function. This action may activate the follow control, causing the system to synchronize the viewing parameters between the two practitioners. The following practitioner's view may then update in real-time to match the movements and adjustments made by the practitioner being followed. In some embodiments, a practitioner may make other practitioners in the session follow them.

These interactive elements may work in conjunction with the practitioner list 12, slide tray 14, and avatar indicator(s) 22 to create a comprehensive collaborative environment. The combination of these features may enable practitioners to maintain awareness of each other's focus areas and activities, whether viewing the same or different regions of the digital pathology image.

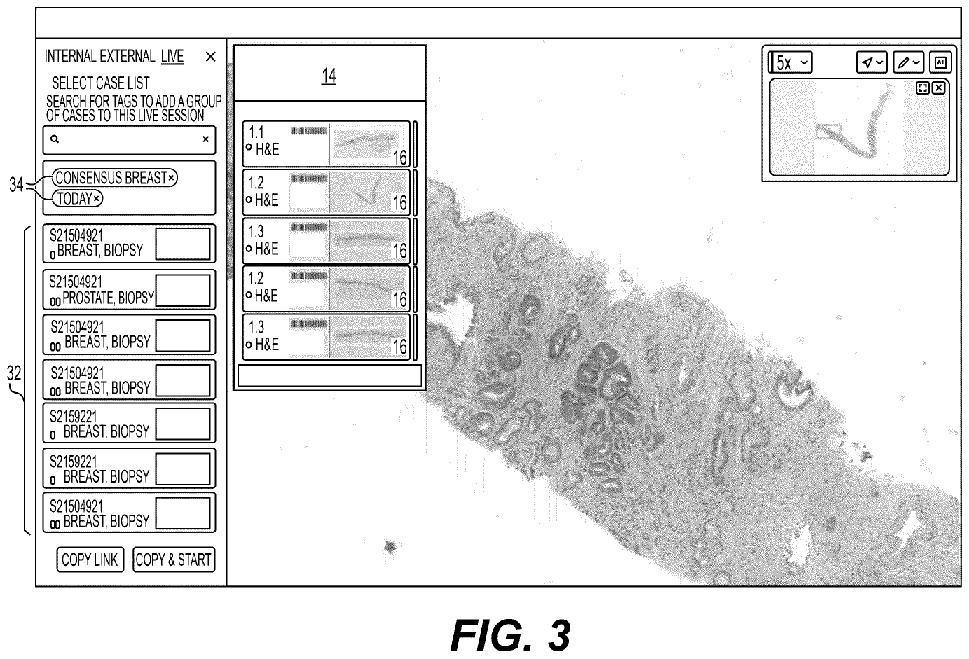

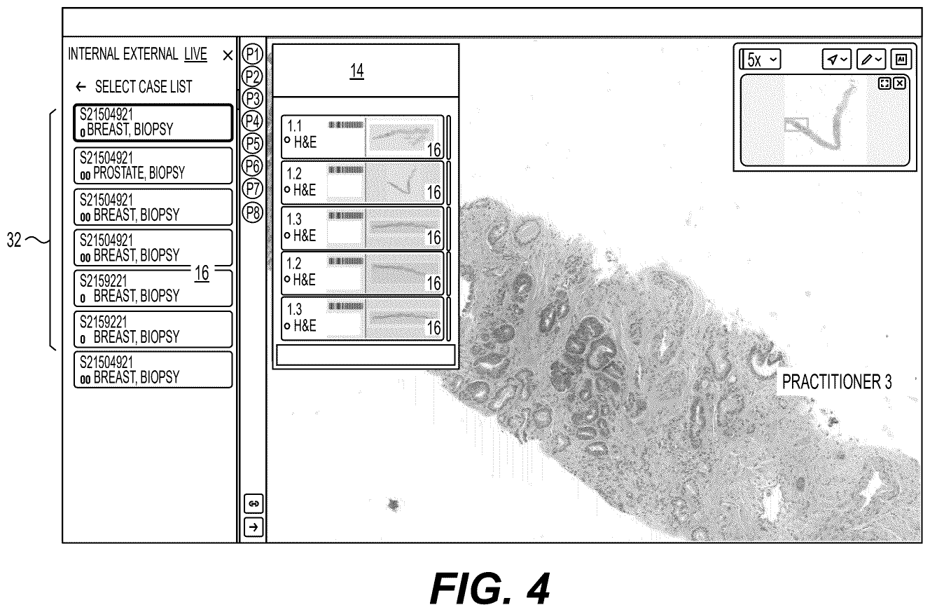

The digital pathology collaboration system may include case management and search functionality to enhance organization and efficiency in collaborative review sessions, as illustrated in FIGS. 3 and 4. These figures depict key components that facilitate the management and selection of cases for review. In some embodiments, a collaboration session may include more than one case. In some instances, there may be a queue of cases 32 for review in a live collaboration session. Cases may be reviewed in one continuous live collaboration session without having to log in and out of the collaborative review session. As can be seen in FIGS. 3 and 4, the queue of cases 32 to be reviewed may be displayed as a list. In follow mode, queue of cases 32 may be mirrored, along with slide tray 14.

In some cases, the system may display tags 34 on user interface 10. Tags 34 may be used to filter and organize cases for review. Tags 34 may represent various attributes or categories associated with the cases, such as tissue type, diagnosis, or date of acquisition. Practitioners may select one or more tags 34 to narrow down the queue of cases 32 displayed for review.

As illustrated in FIGS. 3 and 4, for example below tags 34, a queue of cases 32 may be presented. The queue of cases 32 may show multiple cases available for collaborative review. Each entry in the queue of cases 32 may include information such as case identifiers, patient information 18, or other relevant metadata. Queue of cases 32 may be dynamically updated based on the selected tags 34, allowing practitioners to quickly locate specific cases of interest.

In some cases, tags 34 and queue of cases 32, may work in conjunction to streamline the case selection process. For example, a practitioner may select a tag 34 for “breast biopsy,” and the queue of cases 32 may automatically update to display only cases matching that criteria. This functionality may enable efficient organization and retrieval of relevant cases during collaborative review sessions.

The system may allow practitioners to select cases from the queue of cases 32 for review. When a case is selected, the corresponding slides may be loaded into the slide tray 14, and the main viewing area 24 may display the first slide of the selected case. This integration between the case management features and the viewing interface may facilitate seamless transitions between different cases during collaborative sessions.

In some cases, artificial intelligence (AI) or machine learning algorithms may be employed to automatically tag cases for review. For example, an AI system may analyze digital pathology images and assign relevant tags 34 based on detected features or patterns. These automatically generated tags 34 may then be incorporated into the search tags 34, allowing practitioners to leverage AI-assisted categorization in their case selection process. If the user clicks on an area of interest on a slide sample image corresponding to a region of interest, for example a region tagged as corresponding to an area the AI has identified as potentially corresponding to cancer such as breast cancer, the program may automatically load associated slides, either adjacent slides of tissue, or other slides in the same case that have the same or similar tag(s).

The digital pathology collaboration system may include a comprehensive slide viewing interface and case navigation features, as illustrated in FIG. 4. This figure depicts key components that facilitate efficient review and navigation of digital pathology slides and cases during collaborative sessions.

In some cases, the system may include a slide tray 14 window overlay, visible in FIG. 4. The slide tray 14 window may display thumbnail previews of multiple slides associated with the currently selected case. This feature may enable practitioners to quickly navigate between different slides within a case, enhancing efficiency during the review process.

In some cases, the system may support independent navigation, allowing a practitioner to pause following another user and explore independently. This feature may be particularly useful when a practitioner wishes to examine a specific area of interest without disrupting the collaborative session. For example, a practitioner who is following another user's view may activate a pause function, which may temporarily suspend the synchronization of their viewport with the followed user. The practitioner may then navigate, zoom, or pan independently within the digital pathology slide.

A partial follow may also be possible. If a practitioner is following another user's view, they may be able to break the view by scrolling away from the followed view, or deselecting the full follow feature. A partial follow might synchronize the slide being viewed, the queue of cases 32 and/or the slide tray 14, while not synchronizing the zoom, viewing area 24, and/or viewport rotation. Sessions may begin in partial follow mode by default. A practitioner may want to switch to partial follow if initiating an annotation on their own, which they may or may not have permissions to do. Slides may be annotatable by all practitioners participating in the collaboration session, whether in partial or follow, or with follow turned off. Initiating an annotation may cause follow mode to be automatically switched off while the user enters the annotation, and switched back on when the annotation is complete.

The independent navigation feature may allow practitioners to maintain their own exploration while still participating in the collaborative session. In some cases, the system may provide visual indicators or controls to toggle between following mode, partial follow, and independent navigation mode, ensuring that practitioners can easily manage their viewing preferences during the collaborative review process.

The digital pathology collaboration system may include additional features and functionalities to enhance the collaborative review process and support comprehensive analysis of digital pathology slides.

In some cases, the system may incorporate a voting function that allows practitioners to vote on specific aspects of the case under review. For example, practitioners may use the voting function to indicate their agreement or disagreement with a proposed diagnosis or to highlight areas of uncertainty. The voting function may provide a structured way for practitioners to express their opinions and facilitate consensus-building during collaborative sessions.

The system may include note-keeping and minutes functionality to record and archive findings from collaborative review sessions. This feature may allow practitioners to document important observations, decisions, and discussions that occur during the review process. In some cases, the system may automatically, using artificial intelligence, generate a summary of the collaborative session, including key findings, annotations, and voting results. The recorded notes and minutes may be stored for future reference or used to generate reports for clinical or research purposes.

In some cases, the system may integrate artificial intelligence (AI) tools to assist practitioners during collaborative review sessions. These AI tools may include an AI Grid for automated image analysis, an AI toolbox with various algorithms for detecting specific features or patterns, and other AI-powered functionalities. Practitioners may use these AI tools to supplement their analysis, highlight or review AI-highlighted regions of interest, or provide additional insights during the collaborative review process.

The system may support real-time sharing of annotations made by practitioners during collaborative sessions. When a practitioner creates, modifies, or deletes an annotation on a digital pathology slide, the system may immediately update the view for all other practitioners in the session. This real-time annotation sharing may facilitate rapid communication of findings and enable practitioners to collaboratively build a comprehensive understanding of the case under review.

In some cases, the system may include a presentation mode that allows one practitioner to lead the collaborative session. The practitioner in the leading presenter role may control the view of the digital pathology slide for all other participants, including zoom level, pan position, and slide selection. This presentation mode may be particularly useful for educational purposes, guided reviews, or when a senior pathologist needs to direct the attention of other practitioners to specific areas of interest on the slide.

The presentation mode may allow the leading presenter, the current presenter, and/or the meeting organizer to assign the presenter role to another practitioner in the session. This feature may enable seamless transitions between different presenters during a collaborative review, allowing multiple practitioners to take turns leading the discussion or highlighting specific aspects of the case. Upon a switch to a new presenter, the zoom, views, etc., may be synchronized across all practitioners in the session with that of the new presenter.

In some cases, the system may provide synchronized viewports during the presentation mode. When the leading presenter changes the electronic whole slide image or adjusts the viewing parameters, the viewports of all other practitioners in the session may automatically update to match the presenter's view. This synchronization may ensure that all participants are examining the same region of the slide at the same magnification level, facilitating focused discussions and collaborative analysis.

The system may allow practitioners to pause and resume live updates during the presentation mode. This feature may enable individual practitioners to temporarily disengage from the synchronized view to explore other areas of the slide independently, while still maintaining the ability to quickly rejoin the group's shared view when needed.

In some cases, the system may incorporate case and session-level indicators to provide context and status information during collaborative reviews. These indicators may display information such as the current case being reviewed, the active slide, or the status of ongoing collaborative activities. The indicators may help practitioners maintain awareness of the session's progress and context, particularly when reviewing multiple cases or slides within a single collaborative session.

In one technique, WebSocket technology may be used. The WebSocket technology may allow for data sharing including the sharing of user id, active slide id, cursor coordinates, center coordinates of the viewport, magnification of the viewport, and rotation of the viewport. The use of the WebSocket technology may also allow for annotation creation, updating, and/or deletion; tool activation and interactivity; AI output activation and interactivity; and indication of case being reviewed, in multi-case presentation environment.

The digital pathology collaboration system may incorporate selective information sharing capabilities, allowing practitioners to control access to specific case data or slides within the collaborative environment. In some aspects, the system may implement access control mechanisms, such as passcode requirements or multi-factor authentication, to verify the identity of practitioners before granting access to sensitive information. The system may offer granular permission settings, enabling session organizers or case owners to customize which slides, annotations, or patient data are accessible to different practitioners based on their roles or credentials. For example, a primary pathologist may choose to share only specific regions of interest on a slide with consulting specialists, while maintaining full access to the entire case for themselves. In some implementations, the system may include encryption protocols and secure data transmission methods to protect shared information during collaborative sessions. These security measures may help ensure compliance with relevant privacy regulations and maintain the confidentiality of patient data when collaborating across different institutions or networks.

The digital pathology collaboration system may include a chatroom feature that enables real-time text-based communication between practitioners during collaborative review sessions. This chatroom functionality may be integrated directly into the user interface, allowing pathologists to discuss cases, share observations, and ask questions without interrupting their workflow or switching to separate communication tools. The chatroom may support both public discussions visible to all participants in the session and private messaging between specific practitioners. In some aspects, the system may allow users to attach screenshots or annotations from the digital pathology slides directly to their chat messages, facilitating clear and precise communication about specific regions of interest. The chatroom feature may also include a searchable message history, enabling practitioners to quickly reference previous discussions or findings related to the current case under review.

The digital pathology collaboration system may incorporate audio streaming capabilities to enhance real-time communication between practitioners during collaborative review sessions. This audio functionality may be integrated seamlessly with the existing visual and interactive features of the system, allowing pathologists to discuss cases verbally while simultaneously examining digital slides and annotations. The audio streaming may support various implementations, such as Voice over IP (VoIP) or WebRTC protocols, to accommodate different network environments and institutional preferences. In some aspects, the system may offer adjustable audio quality settings to optimize performance based on available bandwidth, and may include features such as background noise suppression or acoustic echo cancellation to improve clarity during discussions. The audio streaming may be configured to support both group conversations among all participants in a session and private audio channels between specific practitioners for focused consultations. In some implementations, the system may provide options for recording audio discussions, with appropriate consent mechanisms, to facilitate later review or documentation of key decision points in the collaborative analysis process.

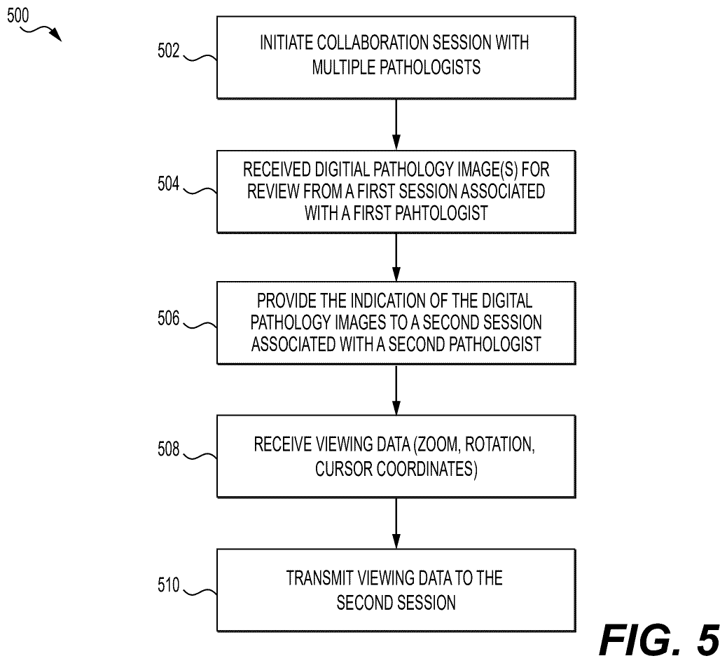

FIG. 5 illustrates a flowchart of a method 500 for collaborative review in digital pathology. The method 500 begins with step 502, where a collaboration session may be initiated between multiple pathologists or other practitioners. In step 504, digital pathology image(s) may be received for review from a first session associated with a first pathologist. In step 506, the indication of the digital pathology images may be provided to a second session associated with a second pathologist. In step 508, viewing data may be received. This viewing data may include zoom level, rotation, and cursor coordinates. In step 510, the viewing data is transmitted to the second session.

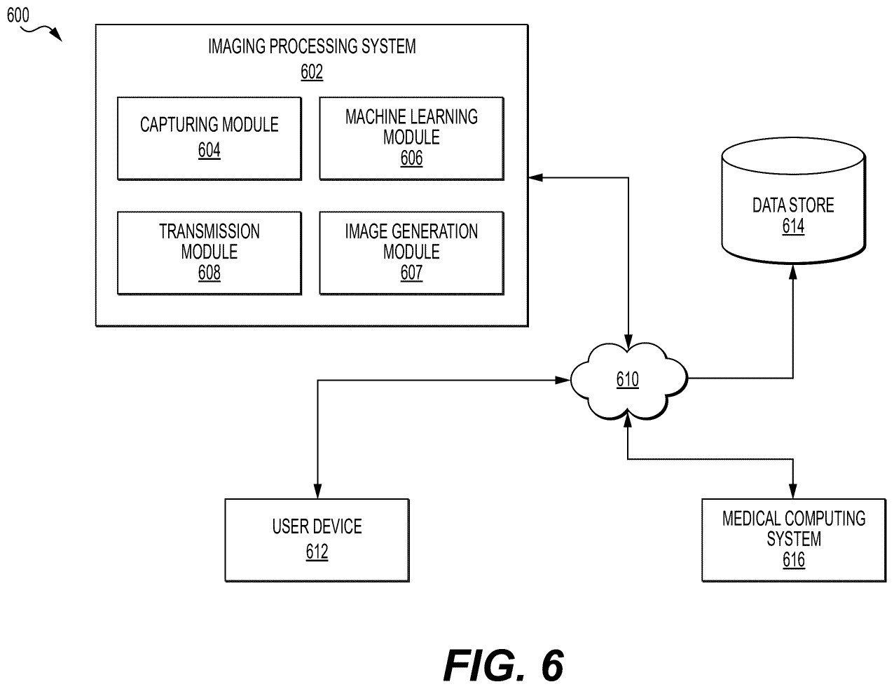

FIG. 6 depicts an exemplary environment 600 that may be utilized with techniques presented herein. One or more user device(s) 612 may communicate across an electronic network 610. The one or more user device(s) 612 may be associated with a user, e.g., a user that is viewing and/or interacting with a generated navigable three-dimensional image, an administrator of one or more components of environment 600, and/or the like. As will be discussed in further detail below, one or more computing system(s) 602 may communicate with one or more of the other components of the environment 600 across electronic network 610.

The user device(s) 612 may be configured to enable a user to access and/or interact with other systems in the environment 600. For example, the user device(s) 612 may each be a computer system such as, for example, a desktop computer, a mobile device, a tablet, an augmented/virtual/extended reality device, and etc. In some embodiments, the user device(s) 612 may include one or more electronic application(s), e.g., a program, plugin, browser extension, etc., installed on a memory of the user device(s) 612. In some embodiments, the electronic application(s) may be associated with one or more of the other components in the environment 600. For example, the electronic application(s) may include one or more of system control software, system monitoring software, software development tools, etc.

In various embodiments, the environment 600 may include a data store 614 (e.g., database). The data store 614 may include a server system and/or a data storage system such as computer-readable memory such as a hard drive, flash drive, disk, etc. In some embodiments, the data store 614 includes and/or interacts with an application programming interface for exchanging data to other systems, e.g., one or more of the other components of the environment. The data store 614 may include and/or act as a repository or source for storing image data, whole slide images (WSI), a generated three-dimensional image, patient data, output data (e.g., from a machine-learning model), and the like (e.g., to be provided/transmitted to user device 612 or to/from any of the other components of environment 600).

In some embodiments, the components of the environment 600 are associated with a common entity, e.g., a service provider, an account provider, or the like. For example, in some embodiments, image processing system (computing system) 602, data store 614, and medical computing system 616 may be associated with a common entity. In some embodiments, one or more of the components of the environment may be associated with a different entity than another. For example, computing system 602 may be associated with a first entity (e.g., a service provider) while medical computing system 616 may be associated with a second entity (e.g., a medical institution or provider). The systems and devices of the environment 600 may communicate in any arrangement. As will be discussed herein, systems and/or devices of the environment 600 may communicate in order to one or more of generate, train, or use a machine-learning model to process imaging data, among other activities.

As discussed in further detail below, the computing system(s) 602 may, one or more of, (i) generate, store, train, communicate with, or use a machine-learning model configured to process imaging data. The computing system(s) 602 may include a machine-learning model and/or instructions associated with the machine-learning model, e.g., instructions for generating a machine-learning model, training the machine-learning model, using the machine-learning model etc. The computing system(s) 602 may include instructions for retrieving data, adjusting data, e.g., based on the output of the machine-learning model, and/or operating a display of the user device(s) 612 to output generated responses to input, e.g., as adjusted based on the machine-learning model. The computing system(s) 602 may include training data, e.g., image data, and may include ground truth, e.g., (i) training whole slide images and (ii) training three-dimensional images to generate a navigable three-dimensional image.

As depicted in FIG. 6, computing system(s) 602 may include capturing module 604. In various embodiments, capturing module 604 is configured to receive a plurality of whole slide images (WSI) associated with a tissue sample. The whole slide images and/or associated data may be gathered and/or compiled by the computing system 602 or using components separate from environment 600. In examples, capturing module 604 may receive the whole slide images from medical computing system 616 via network 610. Medical computing system 616 may be a user device associated with a medial institution, a medical imaging device, or the like. A medical imaging device implementing medical computing system 616 may include image processing system 602, or image processing system 602 may be a separate component from medical computing system 616. A plurality of images (e.g., digital or electronic image or a whole slide image (WSI)) may be received into electronic storage (e.g., cloud-based storage, hard disk, RAM, etc.) such as data store 614. Further, and in various embodiments, capturing module 604 may receive patient data. In examples, patient data may include medical records, demographic information, medical predispositions, diagnoses and the like. Such patient data may be received by capturing module 604 from data store 614, medical computing system 616, user device 612, or the like.

In example, such image data and patient data may be provided to one or more image processing machine-learning models. The one or more image processing machine-learning models may be implemented, generated, trained, or the like by machine-learning module 606. The one or more image processing machine-learning models may be trained based on training data that includes historical/genuine/prior patient tissue images and/or simulated/synthetic image data, historical or simulated patient data, and/or the like. Synthetic image generation may use techniques described in U.S. application Ser. No. 17/645,197, which is incorporated herein by reference. The training data may be used to train the image processing machine-learning models by modifying one or more weights, layers, synapses, biases, and/or the like of the image processing machine-learning models, in accordance with a machine-learning algorithm, as discussed herein. Alternatively, or in addition, such image data may be used to generate a three-dimensional image.

Computing system(s) 602 may also include image generation module 607. In various embodiments, image generation module 607 may be configured to generate a navigable three-dimensional image of a tissue sample based on an output of the one or more machine-learning models. In various embodiments, image generation module 607 may also be configured to generate an interactive display that incorporates the navigable three-dimensional image. In examples, the interactive display enables a user to navigate aspects of the three-dimensional image (e.g., zoom in/out, rotate, flip, view a cross-section, “peel back” layers of the three-dimensional image to view interior aspects, and the like). In further examples, the interactive display that incorporates the navigable three-dimensional image may be operable and/or configured to enable a user to navigate sample levels (e.g., tissue depths of the tissue sample associated with the image(s). Each level may be associated with a WSI.). In other various embodiments, image generation module 607 may be configured to generate a side-by-side display incorporating graphical representations of two or more images (e.g., whole slide images). In various additional embodiments, image generation module 607 may be configured to place a set of whole slide images in an order based an output of a machine-learning model, and may be further configured to “stitch” the whole slide images together based on the ordering.

As depicted in FIG. 6, computing system(s) 602 may also include transmission module 608. In various embodiments, transmission module 608 may be configured to transmit the interactive display, the side-by-side display, and/or the generated navigable three-dimensional image to a user interface, such as of user device(s) 612. For example, the transmission module 608 may coordinate and transmit the user interfaces of the practitioners according to techniques presented herein. In further embodiments, transmission module 608 may be further configured to transmit data from the aforementioned to data store 614 (e.g., for storage or retention), or to medical computing system 616 (e.g., for storage, display, further processing, or the like).

As depicted in FIG. 6, environment 600 may also include electronic network 610. In various embodiments, the electronic network 610 may be a wide area network (“WAN”), a local area network (“LAN”), personal area network (“PAN”), or the like. In some embodiments, electronic network 610 includes the Internet, and information and data provided between various systems occurs online. “Online” may mean connecting to or accessing source data or information from a location remote from other devices or networks coupled to the Internet. Alternatively, “online” may refer to connecting or accessing an electronic network (wired or wireless) via a mobile communications network or device. The Internet is a worldwide system of computer networks-a network of networks in which a party at one computer or other device connected to the network can obtain information from any other computer and communicate with parties of other computers or devices. The most widely used part of the Internet is the World Wide Web (often-abbreviated “WWW” or called “the Web”). A “website page” generally encompasses a location, data store, or the like that is, for example, hosted and/or operated by a computer system so as to be accessible online, and that may include data configured to cause a program such as a web browser to perform operations such as send, receive, or process data, generate a visual display and/or an interactive interface, or the like.

Although depicted as separate components in FIG. 6, it should be understood that a component or portion of a component in the environment 600 may, in some embodiments, be integrated with or incorporated into one or more other components. In another example, the computing system 602 may be integrated in a data storage system. The data storage system may be configured to communicate and/or receive/send data across electronic network 610 to other components of environment 600. In some embodiments, operations or aspects of one or more of the components discussed above may be distributed amongst one or more other components. Any suitable arrangement and/or integration of the various systems and devices of the environment 600 may be used.

It should be understood that in various embodiments, various components of the environment 600 discussed above may execute instructions or perform acts including the acts discussed above. An act performed by a device may be considered to be performed by a processor, actuator, or the like associated with that device. Further, it should be understood that in various embodiments, various steps may be added, omitted, and/or rearranged in any suitable manner.

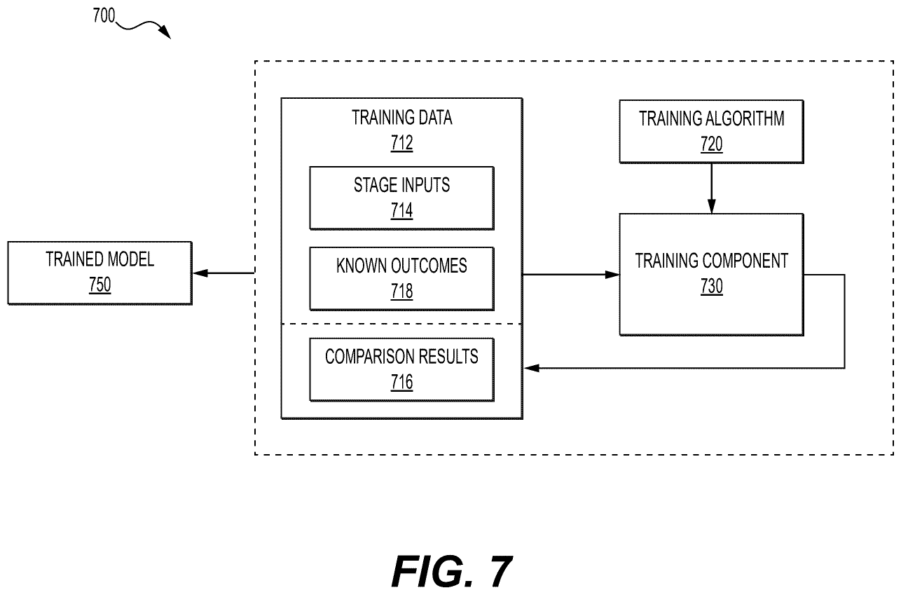

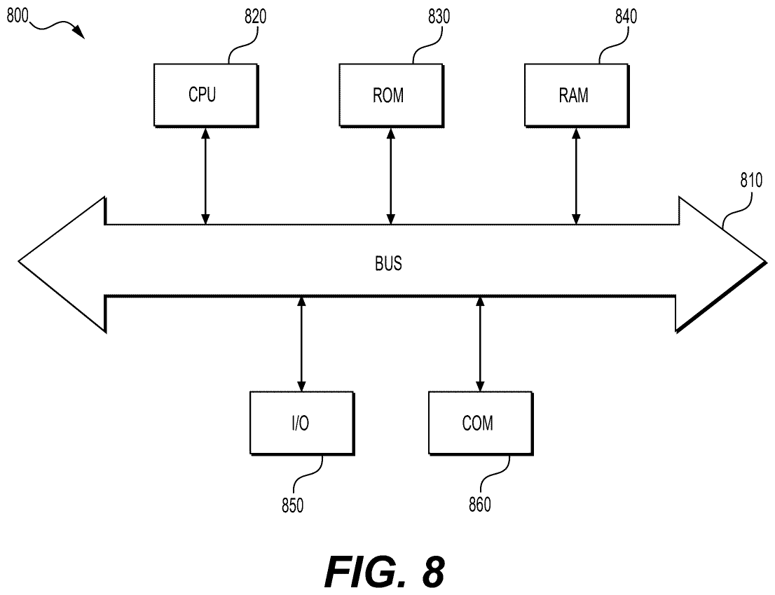

FIG. 7 depicts a flow diagram for training a machine-learning model. As shown in flow diagram 700 of FIG. 8, training data 712 may include one or more of storage inputs 714 and known outcomes 718 related to a machine-learning model to be trained. The storage inputs 714 may be from any applicable source including a component or set shown in the figures provided herein. The known outcomes 718 may be included for machine-learning models generated based on supervised or semi-supervised training. An unsupervised machine-learning model might not be trained using known outcomes 718. Known outcomes 718 may include known or desired outputs for future inputs similar to or in the same category as storage inputs 714 that do not have corresponding known outputs.

The training data 712 and a training algorithm 720 may be provided to a training component 730 that may apply the training data 712 to the training algorithm 720 to generate at least one trained machine-learning model 750, which may execute AI techniques discussed herein. According to an implementation, the training component 730 may be provided comparison results 716 that compare a previous output of the corresponding machine-learning model to apply the previous result to re-train the machine-learning model. The comparison results 716 may be used by the training component 730 to update the corresponding machine-learning model. The training algorithm 720 may utilize machine-learning networks and/or models including, but not limited to a deep learning network such as Graph Neural Networks (GNN), Deep Neural Networks (DNN), Convolutional Neural Networks (CNN), Fully Convolutional Networks (FCN) and Recurrent Neural Networks (RCN), probabilistic models such as Bayesian Networks and Graphical Models, and/or discriminative models such as Decision Forests and maximum margin methods, or the like. The output of the flow diagram 700 may be a trained machine-learning model 750.

A machine-learning model disclosed herein may be trained by adjusting one or more weights, layers, and/or biases during a training phase. During the training phase, historical or simulated data may be provided as inputs to the model. The model may adjust one or more of its weights, layers, and/or biases based on such historical or simulated information. The adjusted weights, layers, and/or biases may be configured in a production version of the machine-learning model (e.g., a trained model) based on the training. Once trained, the machine-learning model may output machine-learning model outputs in accordance with the subject matter disclosed herein. According to an implementation, one or more machine-learning models disclosed herein may continuously be updated based on feedback associated with use or implementation of the machine-learning model outputs.

It should be understood that aspects in this disclosure are exemplary only, and that other aspects may include various combinations of features from other aspects, as well as additional or fewer features.

In general, any process or operation discussed in this disclosure that is understood to be computer-implementable, such as the processes illustrated in the flowcharts disclosed herein, may be performed by one or more processors of a computer system, such as any of the systems or devices in the exemplary environments disclosed herein, as described above. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (CPU), a graphics processing unit (GPU), or any suitable types of processing unit.

A computer system, such as a system or device implementing a process or operation in the examples above, may include one or more computing devices, such as one or more of the systems or devices disclosed herein. One or more processors of a computer system may be included in a single computing device or distributed among a plurality of computing devices. A memory of the computer system may include the respective memory of each computing device of the plurality of computing devices.

As shown in FIG. 8, device 800 may include a central processing unit (CPU) 520. CPU 820 may be any type of processor device including, for example, any type of special purpose or a general-purpose microprocessor device. As will be appreciated by persons skilled in the relevant art, CPU 820 also may be a single processor in a multi-core/multiprocessor system, such system operating alone, or in a cluster of computing devices operating in a cluster or server farm. CPU 820 may be connected to a data communication infrastructure 810, for example a bus, message queue, network, or multi-core message-passing scheme.

Device 800 may also include a main memory 840, for example, random access memory (RAM), and also may include a secondary memory 830. Secondary memory 830, e.g. a read-only memory (ROM), may be, for example, a hard disk drive or a removable storage drive. Such a removable storage drive may comprise, for example, a floppy disk drive, a magnetic tape drive, an optical disk drive, a flash memory, or the like. The removable storage drive in this example reads from and/or writes to a removable storage unit in a well-known manner. The removable storage may comprise a floppy disk, magnetic tape, optical disk, etc., which is read by and written to by the removable storage drive. As will be appreciated by persons skilled in the relevant art, such a removable storage unit generally includes a computer usable storage medium having stored therein computer software and/or data.

In alternative implementations, secondary memory 830 may include similar means for allowing computer programs or other instructions to be loaded into device 800. Examples of such means may include a program cartridge and cartridge interface (such as that found in video game devices), a removable memory chip (such as an EPROM or PROM) and associated socket, and other removable storage units and interfaces, which allow software and data to be transferred from a removable storage unit to device 800.

Device 800 also may include a communications interface (“COM”) 860. Communications interface 860 allows software and data to be transferred between device 800 and external devices. Communications interface 860 may include a modem, a network interface (such as an Ethernet card), a communications port, a PCMCIA slot and card, or the like. Software and data transferred via communication interface 860 may be in the form of signals, which may be electronic, electromagnetic, optical or other signals capable of being received by communication interface 860. These signals may be provided to communication interface 860 via a communications path of device 800, which may be implemented using, for example, wire or cable, fiber optics, a phone line, a cellular phone link, an RF link or other communications channels.

The hardware elements, operating systems, and programming languages of such equipment are conventional in nature, and it is presumed that those skilled in the art are adequately familiar therewith. Device 800 may also include input and output ports 850 to connect with input and output devices such as keyboards, mice, touchscreens, monitors, displays, etc. Of course, the various server functions may be implemented in a distributed fashion on a number of similar platforms, to distribute the processing load. Alternatively, the servers may be implemented by appropriate programming of one computer hardware platform.

Throughout this disclosure, references to components or modules generally refer to items that logically may be grouped together to perform a function or group of related functions. Like reference numerals are generally intended to refer to the same or similar components. Components and/or modules may be implemented in software, hardware, or a combination of software and/or hardware.

The tools, modules, and/or functions described above may be performed by one or more processors. “Storage” type media may include any or all of the tangible memory of the computers, processors or the like, or associated modules thereof, such as various semiconductor memories, tape drives, disk drives and the like, which may provide non-transitory storage at any time for software programming.

Software may be communicated through the Internet, a cloud service provider, or other telecommunication networks. For example, communications may enable loading software from one computer or processor into another. As used herein, unless restricted to non-transitory, tangible “storage” media, terms such as computer or machine “readable medium” refer to any medium that participates in providing instructions to a processor for execution.

One or more techniques presented herein may enable a user, to better interact with a digital image of a glass slide that may be presented on a screen, in a virtual reality environment, in an augmented reality environment, or via some other form of visual display. One or more techniques presented herein may enable a natural interaction closer to traditional microscopy with less fatigue than using a mouse, keyboard, and/or other similar standard computer input devices.

The controllers disclosed herein may be comfortable for a user to control. The controllers disclosed herein may be implemented anywhere that digital healthcare is practiced, namely in hospitals, clinics, labs, and satellite or home offices. Standard technology may facilitate connections between input devices and computers (USB ports, Bluetooth (wireless), etc.) and may include customer drivers and software for programming, calibrating, and allowing inputs from the device to be received properly by a computer and visualization software.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While the disclosed methods, devices, and systems are described with exemplary reference to transmitting data, it should be appreciated that the disclosed aspects may be applicable to any environment, such as a desktop or laptop computer, an automobile entertainment system, a home entertainment system, etc. Also, the disclosed aspects may be applicable to any type of Internet protocol.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

1. A method for collaborative review of digital pathology images, comprising:
initiating a collaboration session including a plurality of practitioners on an electronic network;
receiving an indication of one or more digital pathology images to be reviewed in the collaboration session from a first session instance associated with a first practitioner of the plurality of practitioners;
providing the indication of the one or more digital pathology images to at least a second session instance associated with a second practitioner of the plurality of practitioners;
receiving viewing data comprising zoom level, viewport rotation, and cursor coordinates associated with the first session instance, the viewing data not comprising video data; and
providing the viewing data to at least the second session instance.

2. The method of claim 1, wherein the viewing data further comprises annotation data, and the second session instance displays the cursor coordinates and annotation data, but not the zoom level or viewport rotation.

3. The method of claim 2, wherein the annotation data comprises real-time updates of annotations made by the first practitioner during the collaboration session.

4. The method of claim 1, wherein the viewing data comprising zoom level, viewport rotation, and cursor coordinates is used to reconstruct a view of the first session instance within the second session instance.

5. The method of claim 1, wherein each practitioner of the plurality of practitioners is associated with an avatar displayed within the collaboration session, and further comprising:
receiving a selection of a second avatar in the first session instance, the second avatar being associated with the second session instance; and
in response to the selection, providing data indicative of zoom level, viewport rotation, and cursor coordinates associated with the second session instance to the first session instance.

6. The method of claim 1, further comprising:
displaying a case list comprising multiple cases available for review in the collaboration session; and
receiving a selection of a case from the case list, from the first session instance, to be reviewed in the collaboration session.

7. The method of claim 6, wherein the case list is filtered based on tags associated with each case, the tags comprising at least one of date labels, tissue type labels, or diagnostic labels.

8. A system for collaborative review of digital pathology images, comprising:
a processor; and
a memory storing instructions that, when executed by the processor, cause the system to execute the instructions to perform operations comprising:
initiating a collaboration session including a plurality of practitioners;
receiving, from a first client device, an indication of one or more digital pathology images to be reviewed;
transmitting the indication to at least a second client device;
receiving, from the first client device, viewing data comprising zoom level, viewport rotation, and cursor coordinates, the viewing data not comprising video data; and
transmitting the viewing data to at least the second client device.

9. The system of claim 8, wherein the viewing data further comprises annotation data, and the operations further comprise:
transmitting the annotation data to at least the second client device for display of cursor coordinates and annotation data, but not the zoom level or viewport rotation.

10. The system of claim 9, wherein the annotation data comprises real-time updates of annotations made by a first practitioner during the collaboration session.

11. The system of claim 8, wherein the viewing data transmitted to at least the second client device may be used to reconstruct a view of the first client device within the second client device.

12. The system of claim 8, the operations further comprising:
associating each practitioner of the plurality of practitioners with an avatar displayed within the collaboration session;
receiving, from the first client device, a selection of a second avatar associated with the second client device; and
in response to the selection, transmitting data indicative of zoom level, viewport rotation, and cursor coordinates associated with the second client device to the first client device.

13. The system of claim 8, the operations further comprising:
transmitting a case list comprising multiple cases available for review in the collaboration session to the first client device and the second client device; and
receiving a selection of a case from the case list to be reviewed in the collaboration session.

14. The system of claim 13, wherein the case list is filtered based on tags associated with each case, the tags comprising at least one of date labels, tissue type labels, or diagnostic labels.

15. A non-transitory computer-readable storage medium storing instructions that, when executed by a processor, cause the processor to perform a method for collaborative review of digital pathology images, the method comprising:
initiating a collaboration session including a plurality of practitioners on an electronic network;
receiving an indication of one or more digital pathology images to be reviewed in the collaboration session from a first session instance associated with a first practitioner of the plurality of practitioners;
providing the indication of the one or more digital pathology images to at least a second session instance associated with a second practitioner of the plurality of practitioners;
receiving viewing data comprising zoom level, viewport rotation, and cursor coordinates associated with the first session instance, the viewing data not comprising video data; and
providing the viewing data to at least the second session instance.

16. The non-transitory computer-readable storage medium of claim 15, wherein the viewing data further comprises annotation data, and the method further comprises:
transmitting the annotation data to the second session instance for display of cursor coordinates and annotation data, but not the zoom level or viewport rotation.

17. The non-transitory computer-readable storage medium of claim 16, wherein the annotation data comprises real-time updates of annotations made by the first practitioner during the collaboration session.

18. The non-transitory computer-readable storage medium of claim 15, wherein the method further comprises:
associating each practitioner of the plurality of practitioners with an avatar displayed within the collaboration session;
receiving, from the second session instance, a selection of an avatar representing the first practitioner; and
in response to the selection, transmitting data indicative of zoom level, viewport rotation, and cursor coordinates associated with a client device of the first practitioner to the client device associated with the second practitioner.

19. The non-transitory computer-readable storage medium of claim 15, wherein the method further comprises:
providing a case list comprising multiple cases available for review in the collaboration session; and
receiving a selection of a case from the case list to be reviewed in the collaboration session.

20. The non-transitory computer-readable storage medium of claim 19, wherein the case list is filtered based on tags associated with each case, the tags comprising at least one of date labels, tissue type labels, or diagnostic labels.
